(19) United States
(12) Patent Application Publication
(10) Pub. No.: US 2016/0089089 A1
(43) Pub. Date: Mar. 31, 2016

(54) SYSTEMS AND METHODS FOR DIGITAL PREDICTIVE DISEASE EXACERBATION AND PRE-EMPTIVE TREATMENT

(71) Applicant: AEDIO, INC., Brookline, MA (US)

(72) Inventors: Rahul Kakkar, Brookline, MA (US); Cyan Collier, Oxford (GB); Hitesh Sanganee, Timperley (GB)

(21) Appl. No.: 14/863,876

(22) Filed: Sep. 24, 2015

Related U.S. Application Data
(60) Provisional application No. 62/055,070, filed on Sep. 25, 2014.

(51) Int. Cl. A61B 5/00; A61B 5/0205

(52) U.S. Cl. CPC A61B 5/7282; A61B 5/0205; A61B 5/7275; A61B 5/746; A61B 5/0022; A61B 5/02416

(57) ABSTRACT

The system described herein collects patient data passively and non-passively via onboard and external sensors, and combines the data with past clinical history to generate digital biomarkers. The collected data can also be further combined with other data generating systems to more accurately predict disease exacerbations. The system monitors the digital biomarkers in real-time, and can detect a change in the disease state prior to clinical decompensation and suggest pre-emptive intervention. The system enables a patient to be treated early in the clinical timeline when the disease exacerbation is at the subclinical level rather than waiting until the disease exacerbation reaches the clinical level. Acting when the exacerbation is at the subclinical level enables preemptive treatment rather than reactive treatment, which is often more cost effective while improving clinical outcomes. The system is able to make the predictions by detecting subclinical changes in digital biomarkers that are generated from respiratory, cardiac, patient reported symptoms, user behaviors, and environmental triggers.

### CROSS-REFERENCE TO RELATED APPLICATIONS

This application claims priority to U.S. Provisional Patent Application No. 62/055,070 filed on Sep. 25, 2014 and titled “SYSTEMS AND METHODS FOR DIGITAL PREDICTIVE DISEASE EXACERBATION AND PRE-EMPTIVE TREATMENT,” which is herein incorporated by reference in its entirety.

### BACKGROUND OF THE DISCLOSURE

Typically, diseases treatment begins after the clinical manifestation of the disease. While a physician may be able to diagnose a disease prior the disease manifestation, the clinical diagnosis can only be made in a clinical setting. However, once the disease has exacerbated to the point of clinical manifestation, the course of treatment is reactive. Reactive treatment can often be more expensive and less effective than preemptive treatment.

### BRIEF SUMMARY OF THE DISCLOSURE

Systems and method of the present solution are directed to a system and method for predicting disease exacerbation prior to clinical presentation. With the introduction of widespread use of mobile technology, significantly more clinically relevant patient data can be collected for disease diagnosis, treatment, and monitoring. The system described herein enables the collection of sensitive physiological measures once possible only at the hospital bedside. The system uses this new stream of real time data to monitor and make predictions about the evolution of a patient's disease. The system enables temporally precise clinical disease determinations to be made away from the traditional clinical setting. These temporally precise determinations enable the triggering of timely notifications to patients and caretakers, reducing the expense of urgent hospital-based care.

According to one aspect of the disclosure, a system to detect a disease exacerbation includes a wearable device configured to couple to a patient. The wearable device can include a pulse sensor that is configured to measure a pulse of the patient. The pulse sensor can measure the patient's pulse by transmitting a light signal toward the patient and receiving a reflection of the light signal transmitted back from the patient. The wearable device can also include a breath sensor configured to measure a breath of the patient. The wearable device can also include a wireless module that can be configured to communicate data that includes the breath and pulse measurements of the patient detected by the wearable device. The system can also include a server. The server can be configured to receive the data that includes breath and pulse measurements from the wireless module. The server can include a prediction engine. The prediction engine can generate a digital biomarker as a function of the breath and pulse measurements. The digital biomarker measuring a disease state. The prediction engine can also determine if the digital biomarker crosses a corresponding threshold.

In some implementations, the system also includes a digital signal processing (DSP) engine that is configured to analyze the breath measurement to determine an inspiration to expiration ratio. The system can also include a DSP engine that is configured to analyze the breath measurement to determine a breath rate. The DSP engine can be a component of the wearable device or the server.

The wearable device can also include a first microphone and a second microphone to acoustically record the breath of the patient. In some implementations, the second microphone is used for noise cancelation. The breath measurement can be acoustically recorded tracheal breath sounds.

In some implementations, the DSP engine is configured to detect at least one of a cough, a wheeze, an apnea condition, and a use of an inhaler in the data. The predictive agent can incorporate a past clinical history into the digital biomarker. The digital biomarker can be a time series, and the threshold can define an exacerbation point. The predictive agent can generate an alarm signal responsive to determining that the digital biomarker crossed the corresponding threshold.

According to another aspect of the disclosure, a method to detect a disease exacerbation can include measuring, with a pulse sensor of a wearable device, a pulse of a patient by transmitting a light signal toward the patient and receiving a reflection of the light signal transmitted back from the patient. The method can also include measuring, with a breath sensor of the wearable device, a breath of the patient. Data including the breath and pulse measurements of the patient detected by the wearable device can be transmitted by a wireless module. A server can receive the breath and pulse measurements from the wireless module. The method can also include generating, by a prediction engine of the server, a digital biomarker as a function of the breath and pulse measurements. The prediction engine can determine if the digital biomarker crosses a corresponding threshold.

In some implementations, the method can include analyzing the breath measurement to determine an inspiration to expiration ratio and analyzing the breath measurement to determine a breath rate.

The method can include measuring the breath of the patient with a first microphone and a second microphone. The sounds recorded by the microphones can be tracheal breath sounds. The method can also include detecting at least one of a cough, a wheeze, an apnea condition, and a use of an inhaler in the data.

In some implementations, a past clinical history can be incorporated into the digital biomarker. The digital biomarker can include a time series, and the threshold defines an exacerbation point. In some implementations, the method can include generating an alarm signal responsive to determining that the digital biomarker crossed the corresponding threshold.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising client device in communication with server device; in accordance with an implementation of the present disclosure;

FIG. 1B is a block diagram depicting a cloud computing environment comprising client device in communication with cloud service providers; in accordance with an implementation of the present disclosure;

FIGS. 1C and 1D are block diagrams depicting embodiments of computing devices useful in connection with the methods and systems described herein; in accordance with an implementation of the present disclosure;

FIG. 2A illustrates a block diagram of a system for predicting disease exacerbation; in accordance with an implementation of the present disclosure;

FIG. 2B illustrates a block diagram of an example external sensor for use in the system illustrated in FIG. 2A; in accordance with an implementation of the present disclosure;

FIG. 3 illustrates a block diagram of a client device running the exacerbation prediction application for predicting disease exacerbation; in accordance with an implementation of the present disclosure;

FIG. 4 illustrates a block diagram of the components of an example exacerbation prediction server for use in predicting disease exacerbation; in accordance with an implementation of the present disclosure;

FIG. 5 illustrates a graph of an example biomarker changing over time; in accordance with an implementation of the present disclosure; and

FIG. 6 illustrates a flow diagram of an example method for detecting a potential disease exacerbation; in accordance with an implementation of the present disclosure.

### DETAILED DESCRIPTION

For purposes of reading the description of the various embodiments below, the following descriptions of the sections of the specification and their respective contents may be helpful.

Section A describes a network environment and computing environment which may be useful for practicing embodiments described herein.

Section B describes a systems and methods for predicting disease exacerbation.

A. Computing and Network Environment

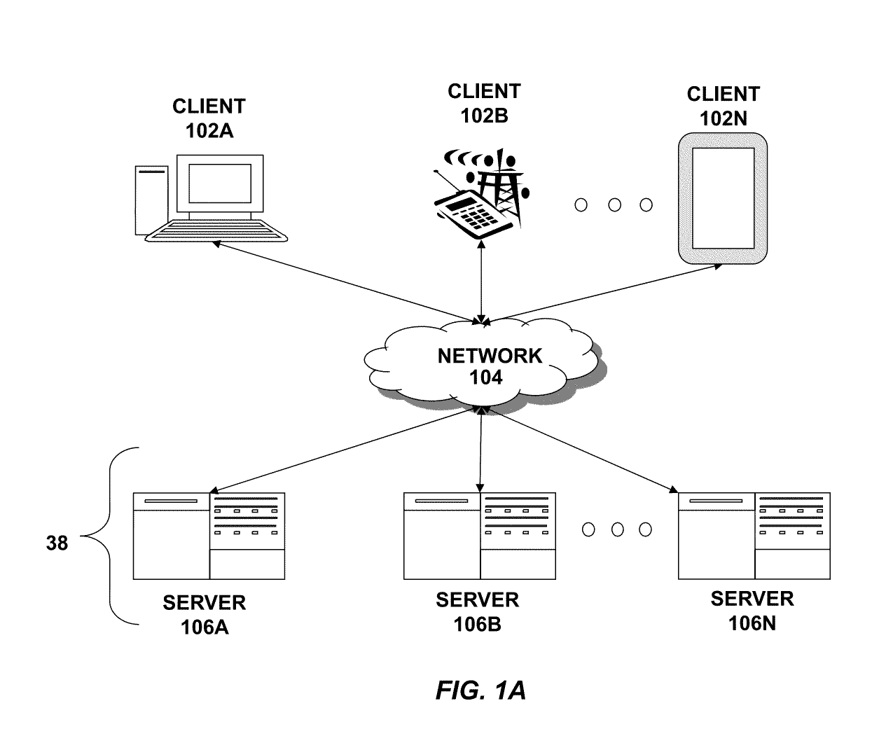

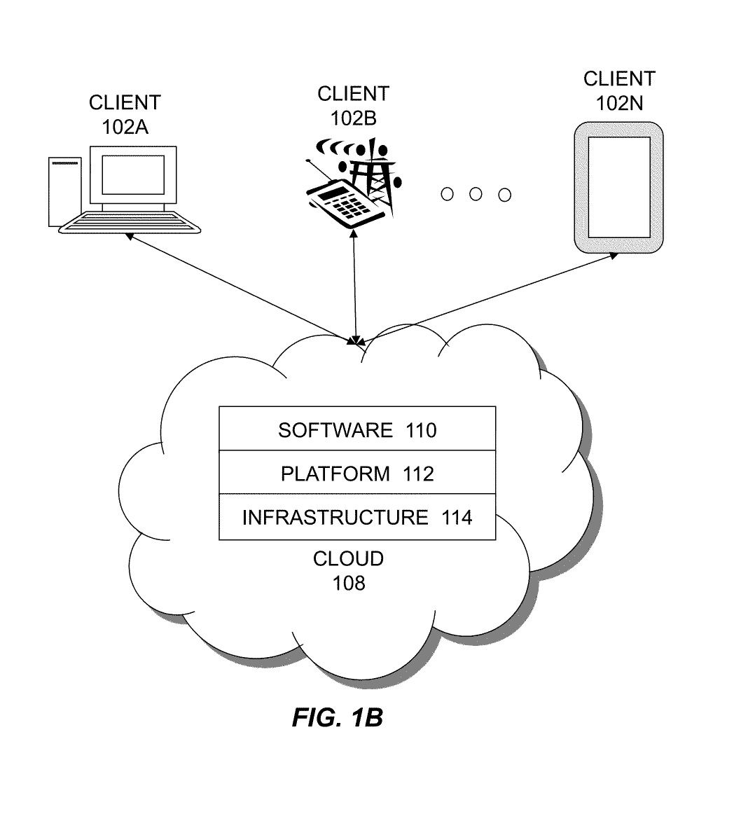

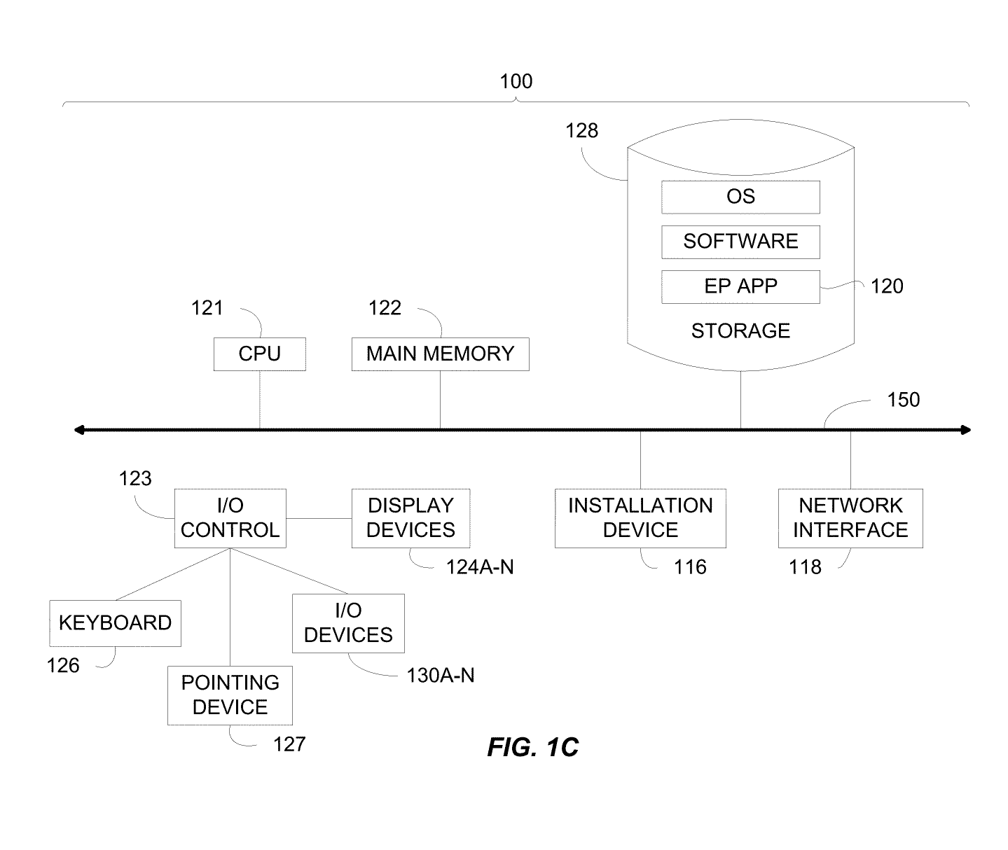

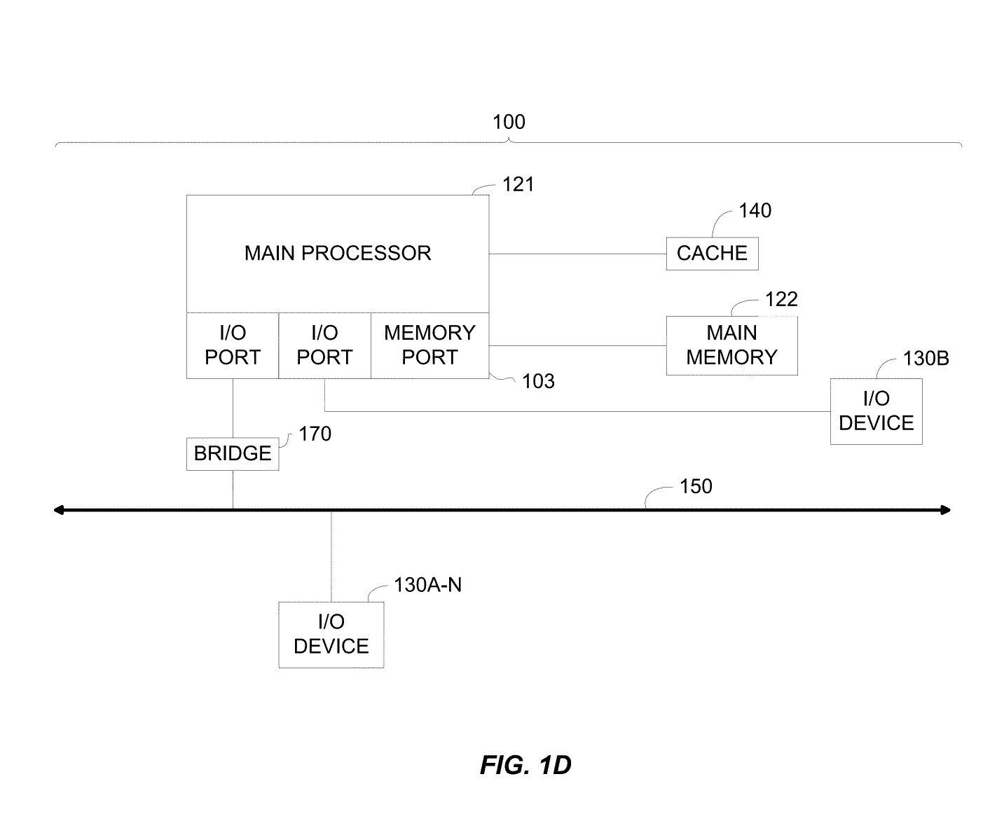

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period.

IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., including Google Fit, and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif. and Apple HealthKit and Samsung Electronics Co of Korea SIMBAND service and Samsung Architecture Multimodal Interactions (S.A.M.I).

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and the software of an exacerbation prediction (EP) application 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including but not limited to, e.g., Microsoft KINECT, Nintendo Wiimote for the WII, Nintendo WII U GAMEPAD, or Apple IPHONE and can be referred to as the “internet of things.” Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software 120 for the experiment tracker system. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via a I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone, smartwatch, or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, RIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is a eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

B. Systems and Methods For Predicting Disease Exacerbation

Systems and methods of the present solution are directed to systems and methods for predicting disease exacerbation prior to clinical presentation. With the introduction of widespread use of mobile technology, significantly more clinically relevant patient data can be collected for disease diagnosis, treatment, and monitoring. The system described herein enables the collection of sensitive physiological measures once possible only at the hospital bedside. The system uses this new stream of real time data to monitor and make predictions about the evolution of a patient's disease. The system enables temporally precise clinical disease decisions to be made away from the clinical setting. These temporally precise decisions enable the triggering of timely notifications to patients and caretakers, reducing the expense of urgent hospital-based care.

As an overview, the system can include a combination of devices, such as an application on a mobile device, smart clothing, or smart watch, that can work in tandem (or independently) to collect patient data via onboard and external sensors, collect patient-reported symptoms, and combine the data with past clinical history and geo-located disease-relevant data to generate digital biomarkers, which may also be referred to as “digicueticals”. The system monitors the digital biomarkers in real-time, and can detect a change in the disease state prior to clinical decompensation and suggest pre-emptive intervention. The system enables a patient to be treated early in the clinical timeline when the disease exacerbation is at the subclinical level rather than waiting until the disease exacerbation reaches the clinical level. Acting when the exacerbation is at the subclinical level enables preemptive treatment rather than reactive treatment, which is often more cost effective while improving clinical outcomes. The system is able to make the predictions by detecting subclinical changes in digital biomarkers that are generated from respiratory, cardiac, patient reported symptoms, user behaviors, and environmental triggers.

While the present solution has been particularly shown and described with reference to specific embodiments, it should be understood by those skilled in the art that various changes in form and detail may be made therein without departing from the spirit and scope of the invention described in this disclosure.

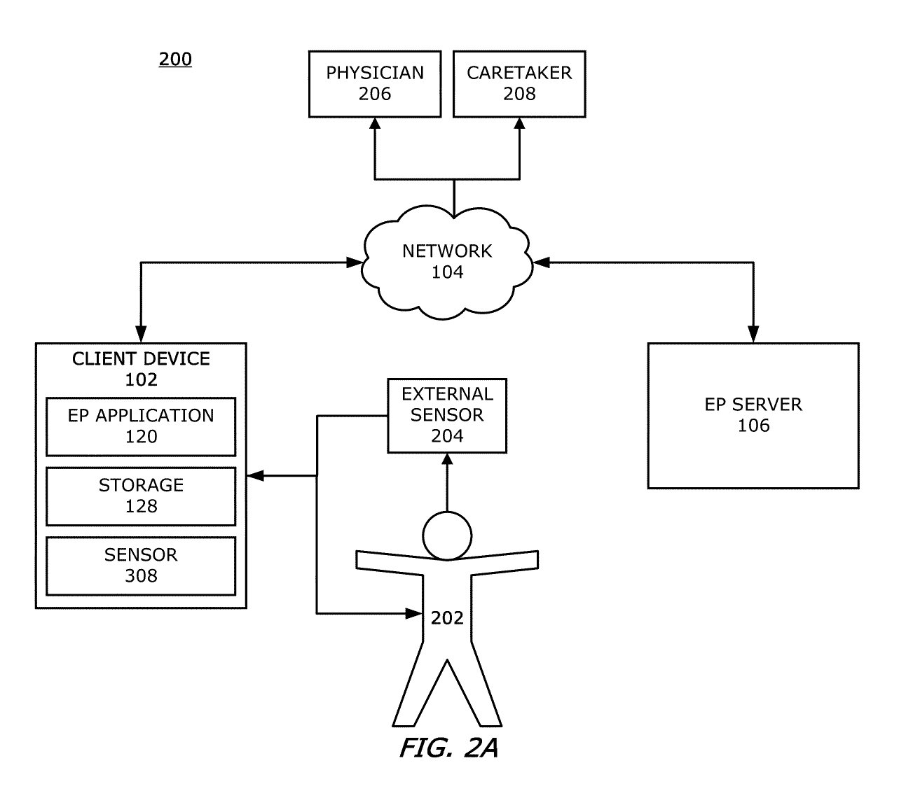

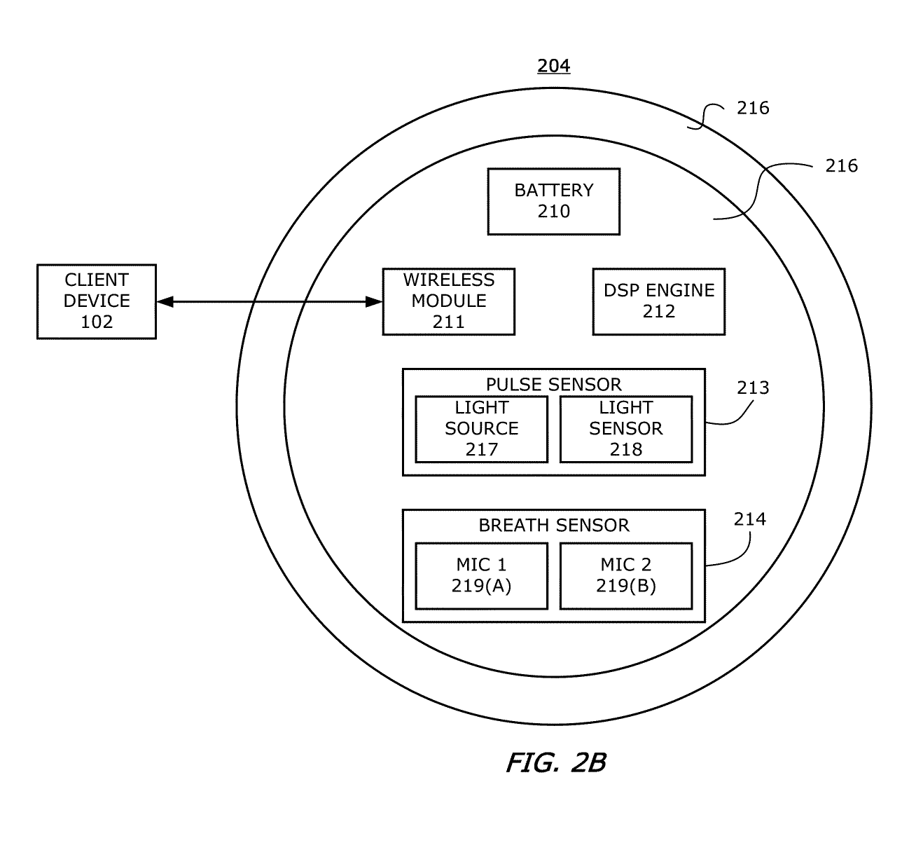

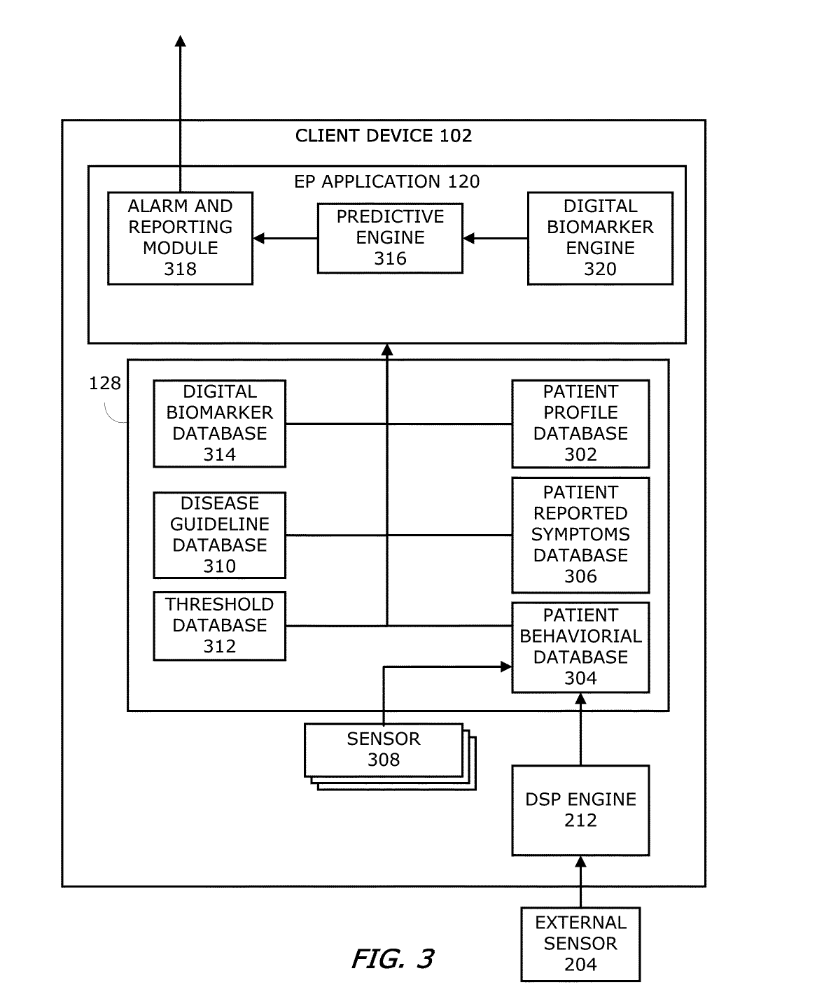

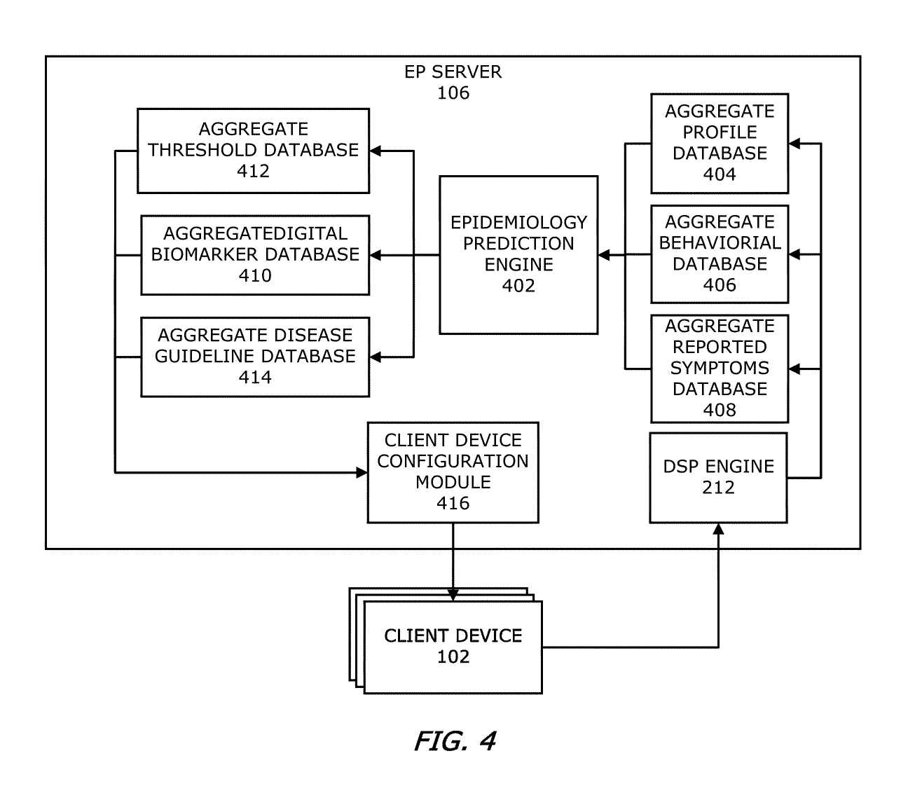

As an overview, FIG. 2A illustrates a block diagram of a system 200 for predicting disease exacerbation. The system 200 can include a client device 102 that communicates with an exacerbation prediction (EP) server 106 (or simply the server 106) over a network 104. The client device 102 can run the EP application 120 and include storage 128 and sensors 308. In some implementations, the EP server 106 can perform the initial configuration of the EP application 120 on the client device 102. A user 202 can interact with the client 102 to provide the client 102 with patient profile information, patient behavioral information, and to report symptoms. In some implementations, additional information is provided to the client 102 via an external sensor 204. The external sensor 204 is external to the client device 102, and can include devices that collect physiological or other data that is provided to the EP application 120. The external sensor 204 can be a heart rate monitor, a scale, a thermometer, or other devices. Using the user 202 provided data, data from the external sensor 204, and data from the sensor 308, the EP application 120 can predict the exacerbation of a disease of the user 202. The client 102, predicting an exacerbation onset, can report back to the user 202, a physician 206, or other caretaker 208 associated with the user 202. Each of the components and functions of the system 200 is described in greater detail in relation to FIGS. 3 and 4. The client device 102, EP application 120, and external sensor 204 are discussed further in relation to FIG. 3 and FIG. 2B, respectively, and the components and functions of the EP server 106 are discussed further in relation to FIG. 4.

FIG. 2B illustrates a block diagram of an example external sensor 204. The sensor 204 can also be referred to as a wearable sensor or a wearable device because, in some implementations, the sensor 204 is coupled to the user 202. The sensor 204 can be a standalone wearable sensor or a component of another device, such as a smart watch, fitness tracker, or similar device. The sensor 204 can include a battery 210, a wireless module 211, and a DSP engine 212. The sensor 204 can also include multiple sensors, such as a pulse sensor 213 and a breath sensor 214.

The components of the sensor 204 can be coupled to a PCB board 215. The PCB board 215 can be a standard, ridged single or multilayer PCB board or the PCB board can be a flexible PCB board that is configured to flex and contour to the shape and movement of the user 202. In some implementations, the sensor 204 can include an adhesive area 216 to enable the sensor 204 to be coupled to the user 202. For example, the sensor 204 can be coupled to the user's neck to enable the sensor 204 to record tracheal breath sounds. The sensor 204 can include a battery 210 that powers the components of the sensor 204. The battery 210 can be a rechargeable battery (e.g., a lithium ion battery) or a replaceable battery (e.g., a coin cell battery). The battery 210 can be periodically recharged by directly coupling the sensor 204 (and battery 210) to a power source. In other implementations, the battery 210 can be changed through wireless induction. For example, the sensor 204 can include induction coils that inductively couple with a wireless power source.

The sensor 204 can also include a wireless module 211. The wireless module 211 is configured to wirelessly communicate with the client device 102, network 104, EP server 106, or any combination thereof. For example, the wireless module 211 can be a 802.11 wireless radio, a bluetooth radio, a zigbee radio, Z-wave radio, cellular radio, or other wireless radio.

The sensor 204 can also include a DSP engine 212. The DSP engine 212 can be a digital signal processor that is configured to preprocess and conditions signals. For example, the DSP engine 212 can process the data signals generated by the pulse sensor 213 and breath sensor 214. The DSP engine 212 can execute an application, program, library, service, task, or any type and form of processor executable instructions. The processor executable instructions executed by the DSP engine 212 are configured to cause the DSP engine 212 to perform signal conditioning that can include filtering the data signals to remove noise or other unwanted signals, up-sampling the data signals, or down-sampling the data signals, or any combination thereof. In some implementations, the DSP engine 212 includes specialty purpose logic that is configured to condition the data signals. For example, the DSP engine 212 may include field-programmable gate arrays (FPGA) or application-specific integrated circuits (ASIC). The DSP engine 212 can analyze the data signals generated by the breath and pulse sensors to identify one or more features of those data signals. For example, the DSP engine 212 can analyze breath measurements to determine an inspiration and expiration ratio; analyze breath measurements to determine a breath rate; and analyze breath measurements to detect at least one of a cough, a wheeze, an apnea condition, and a use of an inhaler.

In some implementations, the DSP engine 212 is configured to perform one or more functions described herein in relation to the client device 102 and the EP server 106, and in some implementations, the client 102 or EP 106 also include a DSP engine 212 that can perform one or more of the functions described in relation to the DSP engine 212 of the sensor 204.

The sensor 204 can also include multiple sensors. The sensor 204 can include a pulse sensor 213, which can include a light source 217 and a light sensor 218. The pulse sensor 213 can detect the pulse of the user 202 by projecting a light toward the user 202 with the light source 217 and then measuring a reflection of the projected light with the light sensor 218. In some implementations, the amount of light reflected back to the light sensor 218 is correlated to the flow of blood through an artery atop which the sensor 204 is placed. The pulsatile flow of the blood correlated to individual heartbeats. In other implementations, the pulse sensor can detect the user's pulse by measuring the electrical activity of the heart. For example, the sensor 204 may be placed on the user's chest and detect electrical activity generated by the contraction of the heart.

The sensor 204 can also include a breath sensor 214. The breath sensor 214 can include multiple microphones 219 (e.g., microphone 219(A) and microphone 219(B)). While two microphones 219 are illustrated, the sensor 204 could include more than two microphones 219 or only a single microphone 219. The microphones 219 are configured to measure tracheal, pulmonary, lung, or other breath sounds. In some implementations, a first microphone 219 can measure the tracheal breath sounds and a second microphone 219 can measure ambient noise, which is used for noise canceling in the audio signal measured by the first microphone 219. The sensor 204 can include an acoustic cavity that directs clinical sounds (e.g., tracheal breath sounds) towards one of the microphones 219. In some implementations, the breath sensor 214 can include a stretch sensor that can detect user breaths by detecting a stretch in the user's chest that occurs with the inspiration of air.

FIG. 3 illustrates the example client 102 in greater detail. The client 102 can include, but is not limited to, a storage device 128 on which a patient profile database 302, a patient behavioral database 304, and a patient reported symptoms database 306 is stored. The data in each of these databases may be entered into the database by a user 202 of the client device 102, a physician 206, a caretaker 208, another user 202, be provided by the client device's internal sensors 308, be provided by the external sensor 204, or be supplied by the server 106. The client 102 can include a DSP engine 212 that preprocesses data received from the sensor 204. One or more processors of the client 102 execute the EP application 120. The EP application 120 may retrieve or be provided data from the databases stored within the storage device 128. The databases stored on the storage device 128 can include a disease guideline database 310, a threshold database 312, and a digital biomarker database 314. Data from the disease guideline database 310, the patient profile database 302, the patient behavioral database 304, the patient reported symptoms database 306, or a combination thereof can be provided to the EP application 120 to make predictions about exacerbations of a disease. The digital biomarker engine 320 may generate or provide digital biomarkers from the data provided to the EP application 120. The predictive engine 316 monitors the generated or provided digital biomarkers by comparing the digital biomarkers to a threshold. In some implementations, the predictive engine 316 determines the user 202 will experience an exacerbation within a predetermined amount of time when the digital biomarker crosses the threshold. The results of the predictive engine 316 can be fed to the alarm and reporting module 318 that can report the results out to the user 202, the physician 206, the other care taker 208, the node 106 or a combination thereof—for example, alarming the user 202 of a disease exacerbation responsive to the predictive engine 316 detecting a biomarker crossing a threshold.

The client device 102 may include any “smart device.” As described above, the client device 102 may include, but is not limited to, smart phones, tablet devices, laptops, and other computational devices. In some implementations, the client device 102 may include other smart devices, such as, but not limited to, smart watches, health and fitness trackers, wearable computers, internet of things devices, and smart clothing. For example, the client device may be a smart watch such as the Moto360 or Apple Watch; a fitness tracker such as a Fitbit or Nike Fuel Band; a wearable computer such as Google Glass; an internet of things device such as a Nest thermostat or other internet enabled device; or smart clothing such as clothing that includes temperature, stretch, or other sensors.

The application 120 may include an application, program, library, service, task or any type and form of executable instructions executable on a device, such as a mobile application executing on a mobile device. The application 120 can make sensitive physiological measurements using the sensors 308, such as over the evolution or course of a disease, data provided by the user 202, and data from other sources to predict the outcomes of a disease course. In some implementations, the other data sources may include, but are not limited to, geo-located, disease-relevant data or environmental data received over the internet. Geo-located, disease-relevant data may include data that patients from within a specific geographic location may be more likely to experience a specific health condition. In some implementations, the data may be collected form a platform provided by a third-part, such as, but not limited to, Google Fit or Apple Health Kit. Other data sources may also include population ethnicity data—for example, that a person of European ancestry is more likely to have cystic fibrosis or that a person of African ancestry is more likely to have sickle-cell anemia. In some implementations, the application 120 can warn the user 202 of the client 102 if the user 202 should seek medical attention. The predictive engine 316 can monitor a disease course in real time and trigger the timely delivery of appropriate outpatient therapy, reducing the expense of urgent and emergent hospital-based care. Through the use of collected data from the client 102, the external sensor 204, and the client device's internal sensors 308, the predictive engine 316 can detect the change in a disease state, before clinical decompensation.

The predictive engine 316 of the application 120 may be designed, constructed and/or configured to make predictions about the exacerbation of a disease based on one or more digital biomarkers. The predictive engine 316 can make predictions by identifying patterns or threshold crossing of the digital biomarkers. The predictive engine 316 can identify the patterns in the digital biomarkers that are provided by the digital biomarker engine 320. The digital biomarkers can include, but is not limited to, physiological time series or other data that alone or in combination can be used to predict the exacerbation of a disease. For example, for an asthmatic patient, digital biomarkers that may be used to predict an asthmatic attack can include the number of times per week that the patient uses a rescue inhaler, the number of times the patient is awoken in a week because of asthmatic related symptoms, environmental temperature, and an indication of air pollutants. The predictive engine 316 may detect threshold crossings of the digital biomarkers to determine if a disease exacerbation will happen within a predetermined amount of time. In some implementations, the predictive engine 316 may use the digital biomarkers as inputs into a machine learning algorithm, such as clustering algorithm, neural network, or a support vector machine, to determine if the user is in or about to enter an exacerbated state.

The digital biomarker engine 320 may be designed, constructed and/or configured to provide one or more digital biomarkers to the predictive engine 316 for a corresponding disease or condition of the user 202. The digital biomarker engine 320 provides the digital biomarkers based on and/or from data received from the disease guideline database 310, the patient profile database 302, the patient behavioral database 304, the patient reported symptoms database 306, or any combination thereof. The digital biomarker engine 320 may determine what data from the above sources is clinically relevant to the user's diseases or conditions, or determine what data improves the predictive outcome of the predictive engine, and provide the selected data to the predictive engine 316. For example, for an asthmatic the digital biomarker engine 320 may determine that the number of times the user 202 uses a rescue inhaler and the atmospheric pollutant count are useful in the prediction of asthma exacerbation, and provide the number of times the user 202 uses the rescue inhaler and the atmospheric pollutant count to the predictive engine 316 as biomarkers. For the same patient, the digital biomarker engine 320 may determine that the user's heart rate, supplied by an external sensor 204, does not provide predictive weight and may not provide the heart rate data to the predictive engine 316 for determining asthma exacerbation. In another example, if the user 202 had a heart condition, the digital biomarker engine 320 may provide the heart rate data to the predictive engine 316 as a biomarker. In some implementations, the digital biomarker engine 320 combines two or more digital biomarkers into an aggregated digital biomarker, such as a score that is a function of each of the digital biomarkers. The digital biomarker database 314 can indicate how the digital biomarker engine 320 should combine and weight the received data to generate an aggregated digital biomarker. The disease guideline database 310 can be a lookup table or other database that indicates what data is clinically relevant for a particular disease or condition. For example, the digital biomarker engine 320 may perform a lookup in the disease guideline database to determine that heart rate is a good biomarker for heart disease but not asthma.

The data provided to the application 120, which is used by the predictive engine 316, can include data from one or more sources. One source of data is the patient profile database 302. The patient profile database 302 can include, but is not limited to, user supplied information, such as past medical history information. Another possible source of data for the predictive engine 316 can be provided by the patient reported symptom database 306. The user 202 may record symptoms, such as severe coughing, shortness of breath, or use of a rescue inhaler and the application 120 may save the data in the patient reported symptom database 306. Another source of data can be the patient behavioral database 304. The patient behavioral database 304 may receive and store data from the sensors 308 and the external sensor 204.

As set forth above, one source of data for the EP application 120 is the patient profile database 302, which is stored in the storage device 128. The patient profile database 302 can include information about the user 202 provided to the EP application 120 from the user 202 or another party, such as the physician 206 or caretaker 208. The data stored in the patient profile database 302 can include, but is not limited to, profile data such as, but not limited to, age, sex, personal disease history, family disease history, current medications, list of previous surgeries or illnesses, place of residency, or other health history information. In some implementations, the information may be provided to the patient profile database 302 by the user 202 when the user registers the EP application 120. In other implementations, the patient profile database 302 may receive data from an electron medical records system connected with the client 102 through the network 104. For example, medical records entered and stored by the user's physician may be automatically retrieved by the EP application 120 using an application programming interface (API).

Another source of data for the EP application 120 may be the patient reported symptoms database 306 stored in the storage device 128. The patient reported symptoms database 306 can be used to store user-entered data about the user's current symptoms or about the user 202 in general. For example, the EP application 120 may request the user 202 take a self-assessment at predetermined or random intervals. The self-assessments can be disease specific and can include, but is not limited to, the Asthma Control Test (ACT) questionnaire, the Minnesota Living with Heart Failure Quotient test. The self-assessments may also be non-disease specific such as a general assessment of function wellness, a questionnaire asking the user 202 to score different symptoms, or a dietary intake questionnaire. The self-assessments may be presented to the user 202 through a graphical user interface (GUI) of the EP application 120. For example, the EP application 120 may at random time intervals present a popup window to the user 202 that asks the user 202 to rank his current, general wellness on a scale of 1 to 10. Other examples of self-reported symptom data that the user 202 can report can include the number of occurrences and severity of a symptoms such as, but not limited to, coughing, wheezing, weakness, use of a rescue inhaler or other medication, user temperature, inability to sleep because of a disease symptom, or the presence and location of pains. For example, the EP application 120 may ask that the user to estimate the number of times in a week that the user 202 had difficulty falling asleep because of troubled breathing.

In another example, the EP application 120 may be designed, constructed and/or configured to allow the user to self-report on events, symptoms, information, related to the user's disease or condition. For example, the EP application 120 may provide a user interface for the user to quickly enter data during or shortly after the occurrence of a symptom. For example, if the user 202 is asthmatic, the EP application 120 may include a button that the user presses if the user 202 uses a rescue inhaler. Pressing the button may automatically record the time the inhaler was used. The EP application 120 may determine a frequency with which the inhaler was used over a given time period and convert this information into a time series that can be fed into the digital biomarker engine 320.

Another source of data for the predictive engine 316 is the patient behavioral database 304 stored in the storage device 128. The data stored in the patient reported symptoms database 306 can be automatically retrieved and stored via one of the onboard client device's internal sensors 308, the external sensor 204, input by the user 202, or a combination thereof. The client device's internal sensors 308 of the client 102 can include, but is not limited to, a microphone, accelerometer, gyroscope, or camera. For example, the accelerometer and the gyroscope may be used to as a pedometer to determine the number of steps the user 202 takes over a given time period. The microphone may be used to measure and record acoustical data such as breath sounds from the user 202. For example, the microphone may be used to determine a breathing rate, recorded as a number of inhale-exhale cycles per minute. The EP application 120 may classify the recorded breath sounds as soft, mild, or hard. The EP application 120 may also identify and characterize various characteristics of the breath sounds, such as bronchial sounds where the EP application 120 determines if the duration of the expiratory sounds and the pitch is as long or longer than the inspiratory sound, broncho vesicular sounds wherein the inspiratory and expiratory sounds are of substantially equal length, but the breath includes full inspiration phase with a softer expiratory phase; crackle sounds of discontinuous, non-musical, brief sounds heard more commonly in inspiration; vesicular sounds of soft and low-pitched that are longer than the expiratory sounds; diminished vesicular breath sounds that are shorter in length than both the inspiratory and expiratory sounds; harsh vesicular breath sounds that are harsh on both the inspiration and expiration; and wheezing breath sounds that can include continuous, high pitched, hissing sounds heard in expiration or also inspiration. In some implementations, the microphone can be used to detect and count coughs, which can be converted into a number of coughs pre time period—for example, number of coughs per hour. Another example client device's internal sensors 308 can include a GPS sensor within the client 102. The GPS sensor can be used to gather and compare location information and to correlate locations with exacerbation patters. For example, the GPS may be used to determine an amount of time spent out of the home each day, or the amount of distance travelled each day. Geolocation may also be used to verify medical facility encounters, such as determining if the user 202 is attending scheduled doctor appointments. In conjunction with the GPS data, the EP application 120 may retrieve information about the user's environment through third party websites. For example, the EP application 120 may access a weather website to determine the temperature and pollen count in the area of the user 202, as indicated by the GPS information.

In some implementations, a microphone is used to record audio sequences of the user's breathing or speech. For example, the EP application 120 can record the nocturnal cardiopulmonary sounds as the user 202 sleeps. Waveform analysis may be performed on the audio sequences. For example, a number of parameters can be derived from mathematical analysis of the audio sequence, such as the mean and peak frequency, frequency entropy (or turbulence), inspiration and expiration decay times and ratio thereof, inspiratory:expiratory duration ratio, or any combination thereof. In some implementations, the recorded auto data may be processed, filtered, or otherwise enhanced. For example, the audio recordings may be high and/or low pass filtered, normalized, amplified, or processed with dynamic range compression.

In some implementations, the EP application 120 may prompt the user 202 to perform specific actions as the EP application 120 records behavioral data. For example, the user 202 may be asked to read aloud a prescribed string of text, such that analysis can be performed on the recording of the user 202 speaking the text. The user may also be asked to walk for a predetermined amount, walk a predetermined distance, or perform an exercise and then record behavioral data, such as heart rate.

In some implementations, the patient behavioral database 304 can receive data from one or more external sensors 204. In some implementations, the external sensor 204 can be a heart rate monitor that can track the user's heart beats per minute (BPM). In some implementations, the heart rate data is combined with other behavioral information. For example, the BPM may be tracked specifically during periods of rest or during periods of physical activity. In other implementations, the external sensor 204 can include a pedometer or other accelerometer based monitor, such as a sleep monitor. The external sensor 204 could also include a scale that wirelessly transmits the user's weight to the EP application 120 after the user 202 users the scale. The external sensor 204 could further include a pulse oximeter to measure the blood oxygenation of the user 202 or a sphygmomanometer to measure the user's blood pressure. The external sensor 204 may communicate with the EP application 120 through a wired or wireless connection. For example, the external sensor 204 may communicate with the EP application 120 through WiFi, a cellular connection, or low energy Bluetooth. The client 102 may be configured to pair with the external sensor 204 and download the data recorded by the external sensor 204 when the external sensor 204 is within range of the client 102. In some implementations, the external sensor 204 can include a microphone external to the client 102. For example, the external microphone may include, but is not limited to, stand-alone microphones, hands free microphones (e.g., Bluetooth headset microphones), and directional microphones.

In some implementations, the external sensor 204 may be external to and/or remote to the user 202 and the client 102. The external sensor 204 may be configured to provide information related to local disease stimulants. For example, the external sensor 204 may be atmospheric sensors at, for example, an airport, and the external sensor 204 may collect atmospheric conditions for the city in which the user 202 is located. The external sensor 204 may store the data in a remote database that the client 102 may connect to via the network 104 to obtain the collected data. The atmospheric conditions can include, but are not limited to, temperature, humidity, pollen count, pollution score, or a combination thereof.

Referring again to FIG. 3, the client 102 includes the disease guideline database 310. The disease guidelines stored in the disease guideline database 310 are clinical guidelines for a range of diseases. For example, the guidelines may be (or be similar to) the guidelines used in guideline-driven management of patients. In some implementations, the guidelines represent to the digital biomarker engine 320 what data (e.g., what data stored in the storage 128) is relevant in determining the current disease state and predicting the disease progression. For example, for an asthmatic patient that is using the client 102 to monitor their asthma, the guideline for asthma may indicate that the digital biomarker engine 320 should combine patient behavior data such as how often have the user 202 had a shortness of breath; how much of the time did the user's asthma keep the user from getting as much done at work, school, or at home; how often does the user's asthma wake the user during the night; and how often does the user need to use an inhaler or nebulizer. The disease guideline database 310 may be implemented as a lookup table that can be referenced by the digital biomarker engine 320. The EP application 120 may access the disease guideline database 310 each time the user 202 indicates a specific disease the user 202 would like to track.

The client 102 can also include a digital biomarker database 314 that provides an indication of how the data the guideline obtained from the disease guideline database 310 should be combined by the digital biomarker engine 320 and analyzed by the predictive engine 316. The digital biomarkers generated by the digital biomarker engine 320 can be used to identify impending disease exacerbation. In some implementations, the data identified as relevant by the disease guideline database 310 can be combined in various ways by the digital biomarker engine 320 depending on one or more factors. For example, if the user 202 indicates that he is experiencing wheezing and coughing associated with asthma once a week and also using a rescue inhaler once a day the digital biomarker database 314 may indicate the two factors should each be given a specific weight when generating a digital biomarker by the digital biomarker engine 320. In this example, if the client 102, through a sensor 308, determines that the patient is not sleeping well or waking multiple times throughout the night, the digital biomarker database 314 may indicate to the digital biomarker engine 320 should weight the use of the rescue inhaler as more important when determining the present disease state of the user 202. The different weights provided to each of the different data streams that are input into the digital biomarker engine 320 can highlight different sensitivities that different population groups may have. For example, the digital biomarker database 314 and the threshold database 312 may indicate to the digital biomarker engine 320 that an African-American male is more likely to suffer from a stroke than a Caucasian male. Accordingly, the digital biomarker database 314 may indicate to the digital biomarker engine 320 that different factors should be weighted differently depending on whether the user 202 is an African-American male or a Caucasian male. In another example, the digital biomarker database 314 may indicate that some data may counteract other data. For example, a digital biomarker may be “improved” (e.g., move further away from a negative threshold) if the user exercises for a predetermined amount of time or logs the consumption of healthy food.

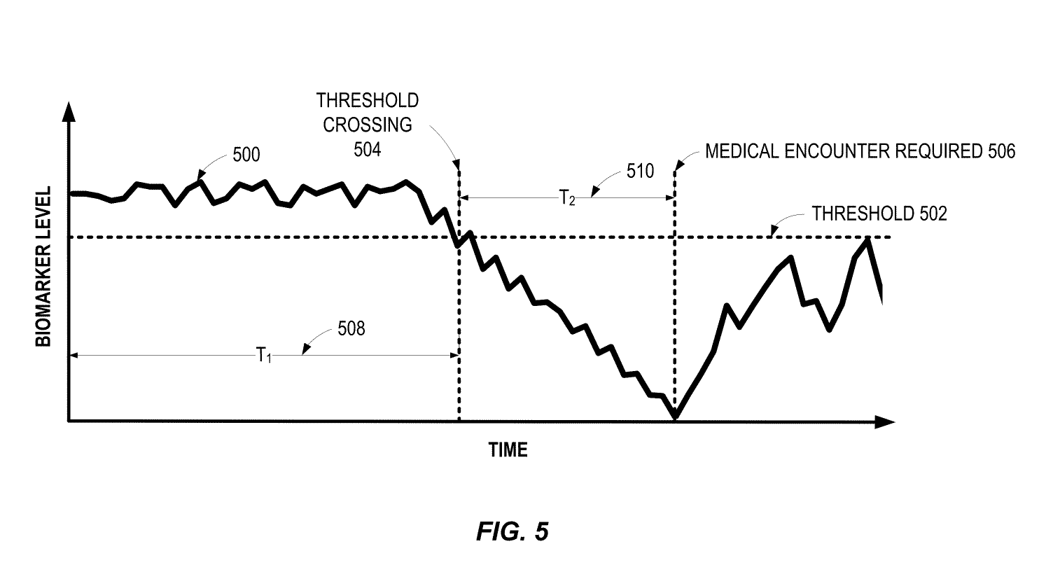

In operation, for example, the predictive engine 316 may receive a digital biomarker from the digital biomarker engine 320 and determine, responsive to a threshold from the threshold database 312, whether an exacerbation in this disease is likely to happen. The predictive engine 316 may monitor the digital biomarker for a threshold crossing. For example, the digital biomarker engine 320 may generate the digital biomarker as a time series. When the digital biomarker crosses the threshold, the predictive engine 316 may determine that a disease exacerbation is likely imminent. The threshold is discussed further in relation to FIG. 5. In some implementations, the data is combined to form a multidimensional variable. In these implementations, machine learning or clustering algorithms may be used to make decisions as to when the user's disease is about to exacerbate. Here the threshold database 312 may provide the number of clusters to use or labelled examples which the learning algorithm of the predictive engine 316 uses to learn or compare the user's data. In some implementations, the threshold database 312 may provide a plurality of thresholds to the predictive engine 316. For example, the threshold database 312 may provide a first threshold that when crossed indicates a mild exacerbation and a second threshold that when crossed indicates a severe exacerbation. In some implementations, the output of the predictive engine 316 is a binary result (e.g., an exacerbation is about to happen or an exacerbation is not about to happen) or a probability range (e.g., an exacerbation is 84% likely to happen within the next 3 days). In some implementations, the predictive engine 316 may continually make predictions or the predictive engine 316 may window data and make predictions on the windowed data. For example, the predictive engine 316 may window the data into one hour windows and make a prediction every one hour. Responsive to determining that a threshold crossing has occurred, the predictive engine 316 may mark a flag or set a bit. The reporting module 318 may monitor the flag and generate a report when the flag is set.

The EP application 120 can also include a reporting module 318. Responsive to the predictive engine 316 making a prediction that the user's disease is about to exacerbate, the reporting module 318 can alert the user. The alert can be sent to the user 202, a care taker, physician, insurance company, pharmacy, or a combination thereof. The alert may include a notification on the client 102, indicating that the user 202 should seek medical attention. The alert could also include sending a text message, push notification, email, or vibration alert. The alert can be sent to the client device 102 or other smart device of the user 202, the care taker, the physician, the insurance company, the pharmacy, or a combination thereof. Example smart devices can includes tablet computers, smart phones, smart watches (e.g., the Simband from Samsung Electronics Co., the Moto360 from Motorola, and the Apple Watch from Apple), smart clothes, or a combination thereof. Smart clothes may include, but are not limited to, items of clothing with embedded electronics and sensors. The reporting module 318 may interface with the scheduling system of a physician's office through an API or other means and may automatically schedule an appointment with the physician if the EP application 120 determines that an exacerbation is imminent. For example, the predictive engine 316 may determine that there is a high likelihood that the user's asthma conditions may worsen over the next few weeks after determining that the user's inhaler is no longer adequately controlling the user's asthma. The reporting module 318 may automatically schedule an appointment with the user's physician to update the user's inhaler prescription. The reporting module 318 may also generate reports that provide an overview of the user's health and disease state. For example, the report may include the user's health trends over the past several weeks or months and enable the user or the user's physician to make quantitative health decisions. For example, the trends may show that while the user's biomarkers did not cross a threshold, the user's biomarkers did consistently worsen when the user did not get at least of a predetermined amount of sleep (e.g., 6.5 hours) a night. In another example, the trends may show that certain food consumption may worsen the user's health state, but does not result in the crossing of a threshold. The trend lines may assist the user in making better health decisions. As described above, the threshold database 312 may provide different thresholds to the predictive engine 316—for example, for a mild and a severe exacerbation. In some implementations, the reporting module 318 may generate a first type of alarm for the crossing of the first threshold and a second type of alarm for the crossing of the second threshold. The reporting module 318 may generate a popup notification when mild threshold is crossed and may automatically schedule an appointment with the physician when a severe threshold is crossed.

FIG. 4 illustrates a block diagram of the components of an embodiment or implementation of an EP server 106. The server 106 can include an epidemiology prediction engine 402. The epidemiology prediction engine 402 can receive inputs from an aggregate profile database 404, an aggregate behavioral database 406, and an aggregate reported symptoms database 408. The epidemiology prediction engine 402 can output data to an aggregate digital biomarker database 410, an aggregate threshold database 412, and an aggregate disease guideline database 414, each of which can act as an input to a client device configuration module 416. The EP server 106 can include a DSP engine 212 that preprocesses data received from the client device 102 or sensor 204.

In some embodiments, the epidemiology prediction engine 402 can include portions or functionality of the predictive engine 316 of the client 102. In some implementations, the epidemiology prediction engine 402 can perform analysis on populations or subgroups of populations rather than individual users. For example, in some implementations, users 202 of client devices 102 may allow their data to be provided back to the server 106 after being cleared of personal information, where the data is provided to one of the respective aggregate profile database 404, aggregate behavioral database 406, and aggregate reported symptoms database 408. The epidemiology prediction engine 402 may make predictions that are used to update the aggregate digital biomarker database 410, the aggregate threshold database 412, and the aggregate disease guideline database 414. For example, as data is reported back to the server 106, the epidemiology prediction engine 402 may determine a connection between cold atmospheric temperatures and a population of user's asthma conditions or find a connection between physical location and heart disease rates. The epidemiology prediction engine 402 may then update the aggregate digital biomarker database 410 to weight of the atmospheric temperature as more important weight when the user 202 is in cold weather.

The server 106 can also include a client device configuration module 416. When the user 202 first registers through the EP application 120, the client 102 may be unconfigured to a specific disease. The client device configuration module 416 may provide relevant information to the EP application 120, such as responsive to an initial questionnaire filled out by the user 202 about what diseases the user 202 would like predictive information about. For example, the client device configuration module 416 may populate the digital biomarker database 314, the disease guideline database 310, and the threshold database 312 responsive to the medical history provided by the user 202. The client device configuration module 416 may provide this information to the client 102 via any type and form of network 104, such as a cellular or WiFi network. In some implementations, as the epidemiology prediction engine 402 updates the aggregate digital biomarker database 410, the aggregate threshold database 412, and the aggregate disease guideline database 414, the client device configuration module 416 may push the updates to the client 102. In other implementations, the updates from the client device configuration module 416 may be made available to the client 102 through user 202 initiated downloads. In some implementations, the user 202 may subscribe to a service or pay for updates to one or all of the aggregate digital biomarker database 410, the aggregate threshold database 412, and the aggregate disease guideline database 414.

FIG. 5 illustrates a graph of an example biomarker 500 changing over time. The graph includes a threshold 502. The biomarker 500 crosses the threshold 502 at a threshold crossing 504. The graph also indicates the time point 506 when a medical encounter may be required by the user. A medical encounter can include, but is not limited to, a trip to the hospital, doctor's office, or pharmacy.

As described above, the biomarker 500 may be the combination of a predetermined number of data samples from the patient profile database 302, the patient behavioral database 304, and the patient reported symptoms database 306. The digital biomarker engine 320 may combine the data, as indicated by the disease guideline database 310 and digital biomarker database 314. The predictive engine 316 may compare the generated biomarker 500 against the threshold 502 that was fetched from the threshold database 312. Referring to FIG. 5, for a time period 508, the user's biomarker 500 is above the threshold 502 and within an acceptable range. When the predictive engine 316 determines that the biomarker 500 crosses the threshold at the threshold crossing 504, the predictive engine 316 can pass an indication to the reporting module 318. The reporting module 318 may then send out an alarm to the user or a care taker of the user. The threshold crossing 504 occurs a time 510 prior to the hospitalization time point 506 time point. Without the predictive warning, the user may have been unaware of his worsening condition, and may not have been made aware of the worsening condition until hospitalization was required. Accordingly, with the warning provided by the EP application 120, the user may be able to seek medical attention prior to the disease exacerbating, enabling for a more clinically effective and more cost effective treatment of the disease.

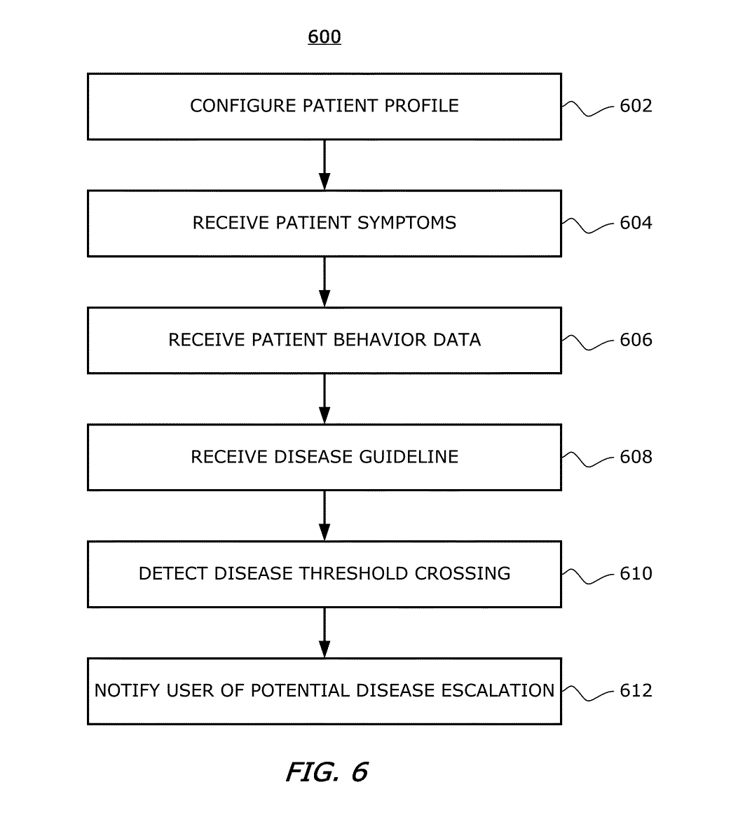

FIG. 6 illustrates a flow diagram of an example embodiment of a method 600 for detecting a potential disease exacerbation. The method 600 can include configuring a patient profile (step 602). An exacerbation prediction application may receive patient symptoms (step 604) and patient behavior data (step 606). The application may also receive a disease guideline (step 608). The application can analyze one or more digital biomarkers to detect a threshold crossing (step 610). Responsive to detecting a threshold crossing, the application can notify the user of a potential disease escalation (step 612).

At step 602, when configuring the patient profile, the user may provide medical history information and information such as sex and age to the EP application 120. The user may provide the medical history information through a GUI of the EP application 120 executing on a mobile device. The user may also log into a website associated with the EP server, and enter the profile information via the website. Some or all of the user's profile may be automatically configured. For example, the EP application 120 may interface with the digital medical records of the user as provided by the user, a hospital, a user's physician, or an insurance provider. The profile information may be stored in the client 102 within the patient profile database 302.

At step 604, the EP application receives the user's symptoms. The user's symptoms may be stored by the EP application 120 in the patient reported symptoms database 306. The user's symptoms may be self-reported symptoms that are related to the user's disease. For example, for a user with asthma, the reported user's symptoms may include the number of times the user uses a rescue inhaler or the number of times the user experiences severe wheezing. The user may self-report the symptoms as they occur, or the EP application 120 may prompt the user to enter symptom information at predetermined or random intervals. For example, for an asthmatic the EP application 120 may randomly request that the user rate their ease of breathing.

At step 606, the EP application 120 receives user behavior data. The user behavior data may be stored in the patient behavioral database 304. The behavior data may include, but is not limited to, data that is collected by the sensors 308 of the client 102 or the behavior data may also include environmental data associated with the user's environment. The behavior data may include, but is not limited to, heart rate data, acoustic data of the user breathing, temperature, pedometer information, and blood pressure information. In some implementations, the behavior data is collected automatically without the user's direct input. For example, accelerometer sensors within the client 102 may collect and count the number of steps that the user takes every day. In other implementations, the user may initiate the collection of data by the sensors of the client 102. For example, the user may initiate an acoustic analysis of the user's breather by recording the user's breathing sounds with a microphone of the client 102 for a predetermined amount of time.

At step 608, a disease guideline associated with the user's disease is received. The disease guideline indicates what data collected by the client 102 is relevant to the predication of the disease exacerbation. The data is combined by the digital biomarker engine to generate a digital biomarker. The digital biomarker engine weighs and combined the weighted data into a digital biomarker. The digital biomarker is provided to the predictive engine 316, which can determine if a disease exacerbation is will occur within a predetermine time frame.

At step 610, the predictive engine 316 analyzes one or more digital biomarkers related to the user's conditions or diseases to detect a threshold crossing. The digital biomarkers can be provided to the predictive engine 316 by the digital biomarker engine 320. The digital biomarker engine 320 can select digital biomarkers (or generate aggregate digital biomarkers) from the self-reported symptoms received from the patient, patient behavior data, the patient's profile data, or a combination thereof. As illustrated in FIG. 5, the digital biomarker may be analyzed over time and compared to a threshold provided to the predictive engine 316 by the threshold database 312. When the predictive engine 316 determines that a threshold crossing has occurred the predictive engine 316 may determine that the user is about to experience a disease exacerbation. In some implementations, the predictive engine 316 uses machine learning to classify the biomarker within a state space. In these implementations, the digital biomarker may not cross a threshold as illustrated in FIG. 5, but rather the biomarker may transition to a new state space or cluster.

At step 612, the EP application 120 notifies the user of an upcoming/impending or potential disease escalation. When the predictive engine 316 determines there is a threshold crossing or a transition from a normal state to a diseased state, the predictive engine 316 may send an indication to the reporting module 318, which notifies the user a disease exacerbation may occur within a predetermined amount of time. The notification may include a push notification or a popup notification to the client 102. The notification could also include, but is not limited to, a telephone call, text message, instant message, email, a vibration alert, or other form of electronic communication to the user or a recipient selected by the user. In some implementations, the notification may be to another system. For example, the notification may be sent to a physician's scheduling software such that an appointment is automatically scheduled for the user.

As an example of the method 600 for a user with asthma, the method may include the user configuring a patient profile. When configuring the patient profile, the user may provide medical history and other information to the application. The user may also indicate that the user wishes to receive predictive information about their asthma condition. Responsive to the user indicating that he would like to receive predictive information about his asthma condition, the application may request additional information related to the user's asthma. For example, the application may ask how many times a week the user uses a rescue inhaler. The application may save the profile information in a patient profile database stored on the user's mobile device.

Once configured, the user may enter patient reported symptoms into the application. For example, if the user begins to wheeze, the user may enter when and for how long the wheezing persisted. In some implementations, the user reported symptoms may be provided to the application in response to the application presenting a questionnaire to the user. For example, for the user with asthma, the application may present the Asthma Control Test questionnaire to the user. The reported symptoms can be stored in a patient reported symptoms database stored on the user's mobile device. The application can also receive patient behavior data from sensors on or associated with the user's mobile device. For example, the user may place the application in an audio recording mode at night. As the user sleeps, the application may use the microphone of the mobile device to detect breather patterns, such as coughing and wheezing. The data collected form the sensors may be stored in a patient behavior database on the mobile device.

As the application receives the data from the sensors or as symptom occurrences are input into the application, the application may select some of the data to use as digital biomarkers that are provided to the predictive engine of the application. For example, for the asthmatic user, the application may use the number of times in the last week that the user's asthma prevented the user from working, the number of time in the last week the user had shortness of breath, the number of times in the past week that the user's asthma woke up the user, the number of times in the last week the user used a rescue inhaler, and a pollutant count of the user's city as biomarkers for exacerbation of the user's asthma.

The application may compare one or more of the biomarkers to one or more thresholds or use the biomarkers as input into a machine learning algorithm to predict an imminent exacerbation of the user's asthma. The application may look for specific patterns or analyze the digital biomarker as time series for threshold crossings. For example, the application may determine that the user's use of a rescue inhaler increased from twice a week to five times a week, which is above a threshold for normal use. The application may also determine that the pollutant count is relatively high for the user's location. The application may determine that based on the pattern of inhaler use crossing the threshold into a higher level of use and the presence of a high pollutant count, the user is likely to experience an exacerbation in their asthma within the next week.

Responsive to the application determining that the user is likely to experience a disease exacerbation, the application can warn the user of the likely exacerbation. The warning may include a text message or a notification on the user's mobile device. The warning may include a notification to a caretaker or a physician. The application may automatically schedule an appointment with the physician such that the user can primitively receive treatment. For example, the application may schedule an appointment with the physician so the physician can examine the user and determine if the user's medication regimen should be altered.

What is claimed:

1. A system to detect a disease exacerbation, the system comprising:
a wearable device configured to couple to a patient, the wearable device comprising:
a pulse sensor configured to measure a pulse of the patient by transmitting a light signal toward the patient and receiving a reflection of the light signal transmitted back from the patient;
a breath sensor configured to measure a breath of the patient;
a wireless module configured to communicate data comprising the breath and pulse measurements of the patient detected by the wearable device;
a server configured to receive the data comprising the breath and pulse measurements from the wireless module, the server comprising a prediction engine, wherein the prediction engine is configured to:
generate a digital biomarker as a function of the breath and pulse measurements, the digital biomarker measuring a disease state; and
determine if the digital biomarker crosses a corresponding threshold.

2. The system of claim 1, further comprising a DSP engine configured to analyze the breath measurement to determine an inspiration to expiration ratio.

3. The system of claim 1, further comprising a DSP engine configured to analyze the breath measurement to determine a breath rate.

4. The system of claim 1, wherein wearable device further comprises a first microphone and a second microphone to acoustically record the breath of the patient.

5. The system of claim 4, wherein the breath measurement is acoustically recorded tracheal breath sounds.

6. The system of claim 1, wherein the DSP engine is configured to detect at least one of a cough, a wheeze, an apnea condition, and a use of an inhaler in the data.

7. The system of claim 1, wherein the predictive agent is configured to incorporate a past clinical history into the digital biomarker.

8. The system of claim 1, wherein the digital biomarker comprises a time series.

9. The system of claim 1, wherein the threshold defines an exacerbation point.

10. The system of claim 1, wherein the predictive agent is configured to generate an alarm signal responsive to determining that the digital biomarker crossed the corresponding threshold.

11. A method to detect a disease exacerbation, the method comprising:
measuring, with a pulse sensor of a wearable device, a pulse of a patient by transmitting a light signal toward the patient and receiving a reflection of the light signal transmitted back from the patient;
measuring, with a breath sensor of the wearable device, a breath of the patient;
transmitting, by a wireless module of the wearable device, data comprising the breath and pulse measurements of the patient detected by the wearable device;
receiving, by a server, the breath and pulse measurements from the wireless module;
generating, by a prediction engine of the server, a digital biomarker as a function of the breath and pulse measurements, the digital biomarker measuring a disease state; and
determining, by the prediction engine of the server, if the digital biomarker crosses a corresponding threshold.

12. The method of claim 11, further comprising analyzing the breath measurement to determine an inspiration to expiration ratio.

13. The method of claim 11, further comprising analyzing the breath measurement to determine a breath rate.

14. The method of claim 11, further comprising measuring the breath of the patient with a first microphone and a second microphone.

15. The method of claim 11, further comprising measuring tracheal breath sounds.

16. The method of claim 11, further comprising detecting at least one of a cough, a wheeze, an apnea condition, and a use of an inhaler in the data.

17. The method of claim 11, further comprising incorporating a past clinical history into the digital biomarker.

18. The method of claim 11, wherein the digital biomarker comprises a time series.

19. The method of claim 11, wherein the threshold defines an exacerbation point.

20. The method of claim 11, further comprising generating an alarm signal responsive to determining that the digital biomarker crossed the corresponding threshold.
